(19) United States
(12) Patent Application Publication
(10) Pub. No.: US 2026/0265656 A1
(43) Pub. Date: Sep. 10, 2026

(54) SYNTHETIC BASEMENT MEMBRANE AND METHODS FOR THE PRODUCTION THEREOF

(71) Applicant: FRAUNHOFER-GESELLSCHAFT ZUR FÖRDERUNG DER ANGEWANDTEN FORSCHUNG E.V., München (DE)

(72) Inventors: Tobias Weigel, Würzburg (DE); Bastian Christ, Würzburg (DE); Christina Neser, Bad Mergentheim (DE); Maximilliane Wußmann, Würzburg (DE); Antje Appelt-Menzel, Dettelbach (DE)

(21) Appl. No.: 19/557,344

(22) Filed: Mar. 5, 2026

(30) Foreign Application Priority Data
Mar. 7, 2025 (DE) 10 2025 108 816.0

(51) Int. Cl. C12M 1/12; A61L 27/22; A61L 27/56; C12M 3/00

(52) U.S. Cl. CPC C12M 25/02; A61L 27/227; A61L 27/56; C12M 21/08

(57) ABSTRACT

The present invention relates to methods for the production of an artificial basement membrane comprising a fiber layer, artificial basement membranes, comprising a fiber layer, in particular produced by means of the method according to the invention as well as in vitro test systems, cell culture systems, filtration systems and organ systems, comprising at least one artificial basement membrane of the present invention.

The present invention relates to methods for the production of an artificial basement membrane comprising a fiber layer, artificial basement membranes, comprising a fiber layer, in particular produced by means of the method according to the invention as well as in vitro test systems, cell culture systems, filtration systems and organ systems, comprising at least one artificial basement membrane of the present invention.

The basement membrane is a cell-free tissue with a thickness of 1 to 3 μm, which is composed of fibrillar as well as reticular structural proteins. The fibrillar structures represent the basic stability of the membrane and are mainly composed of type 3 collagen. Towards the stromal tissue, this structure is anchored to the adjoining tissue via further collagens, such as type 7. On the other side, the basement membrane consists of reticular structural proteins, mainly type 4 collagen and laminins, which form a dense network. This structure mainly has the function of providing specific adhesion sequences in high density for epithelial and endothelial cells. In general, this membrane is permeable for most molecules and macromolecules and can additionally be penetrated by immune cells, such as macrophages as defense against pathogens. The basement membrane thus represents a border tissue, which, together with the cellular component, creates the barrier in each epithelium and endothelium.

The absorption capacity through these barriers is of essential importance in the development of new active pharmaceutical ingredients because the biological availability in the body is essential for the efficacy of the increasingly complex active ingredients. There is thus a steadily increasing demand for human physiological tissue models, which replicates the barrier for transport studies. Barrier models, which map pathological properties, such as infections, genetic diseases or tumors and which are likewise applied in the development of active pharmaceutical ingredients, are likewise developed. At the same time, this is also about reducing animal testing and offering more potent alternatives (on the basis of the 3R principle). The 3R principle stands for reduce, refine and replace and thus for the reduction, the refinement and the replacement of animal testing.

For the production of these tissues, the endothelial or epithelial cells require the defined dense structure of the basement membrane as well as the provision of the adhesion sequences by type 4 collagen and/or different laminins. For these models, the standard method uses “tracked etched” membranes as cell culture surface, which provides a dense surface across its smooth plastic surface and provides for the permeability of culture medium and active ingredients through pores with defined sizes in the range of a few 100 nm up to several micrometers (depending on the application). In order to provide the cell adhesion points, however, these surfaces have to first be coated with proteins or peptides/peptide sequences from the basement membrane, in particular type 4 collagen, laminins and fibronectin. This results in several disadvantages, which primarily relate to the substrate material and the protein coating thereof.

The smooth substrate material with incorporated pores has the disadvantage that molecules only have a small number of penetrations or a small passage surface, respectively, at their disposal after passage through the cellular barrier. The transport of active ingredients through the barrier construct is thus slowed down significantly and is optionally prevented or delayed, respectively, to a large extent and thus leads to an underestimation of the bio-availability of the active ingredient (faulty simulation of the absorption of drugs). In some cases, the hydrophobic surface even has the result that nonspecific molecule adsorption preclude transport studies on the membrane material. In specific applications with further cells below the barrier model, the small number and size of pores likewise reduce cellular interactions between barrier cells and below adjoining stromal cells or immune cells, respectively.

On the one hand, defined protein solutions of type 4 collagen and/or laminins and/or fibronectin, which functionalize the material surface via nonspecific adsorption, serve as coating for this. Especially the elaborate production and purification and the significant costs associated therewith are a disadvantage of defined protein solutions. On the other hand, the functionalization takes place with undefined protein solutions: these solutions are made from animal or human tissues, which have a high percentage of basement membrane proteins. The most widely used material is Matrigel™, which is isolated from artificial tumors in animal testing. For this purpose, artificial tumors are induced into mice and are removed again after the growth thereof, the material is liquefied, purified and subsequently offered as coating solution. A high batch variance is a disadvantage thereby as well as relatively high costs due to elaborate production as well as annual numbers of used animals of approximately 300,000 mice worldwide. Advanced developments, which replace the isolation of the basement membrane proteins in mice with direct human sources are currently made as alternatives. The tissue of the placenta, which is produced during the birth of every human being and which would be available as waste product for preparation and isolation of EZM proteins (extracellular matrix) serves this purpose. However, the batch variance due to genetic or pathological differences of the respective human proteins is also a disadvantage.

It is a further disadvantage of the application of coating solutions for the basement membrane that only the qualitative and quantitative provision of the adhesion sequences can be achieved. The simple principle of the nonspecific adsorption of the proteins thus creates a randomly generated provision of the necessary peptide sequences but not a physiological structural arrangement of these proteins for the structural imitation of the basement membrane.

It is the technical problem, on which the present invention is based, to provide methods for the production of basement membranes and basement membranes produced therewith, which do not have the above-mentioned disadvantages of known basement membranes, in particular those, which make it possible to provide functionally improved in vitro test systems, cell culture systems, filtration systems and organ systems.

This technical problem is solved by means of the provision of the teachings of the independent and dependent claims as well as the corresponding description.

This technical problem is solved in particular by means of a method for the production of an artificial basement membrane comprising a fiber layer, which is embedded in a matrix composed of at least one basement membrane protein, comprising the following method steps:
a) producing and applying a fiber layer, comprising fibers with a fiber diameter of 100 to 2000 nm, by means of electrospinning a fiber material onto the surface of a carrier element arranged in an electrospinner, wherein the fiber layer has a fiber number of 1000 to 30000 fibers/mm²,
b) applying a stabilizing frame of at least one polymer onto the fiber layer arranged on the carrier element under conditions, which allow for an association of the stabilizing frame with the fiber layer,
c) cultivating the fiber layer associated on the stabilizing frame with cells of at least one cell type, which are introduced into the fiber layer and which are capable of expressing at least one basement membrane protein, under conditions, which allow for the expression of the at least one basement membrane protein and by obtaining a matrix, which is composed of the at least one basement membrane protein and in which the fiber layer is embedded and
d) removing the cells of the at least one cell type by obtaining the artificial basement membrane.

The present invention accordingly provides that a fiber layer is produced in a first method step a) by means of electrospinning, wherein this fiber layer has fibers with a fiber diameter of 100 to 2000 nm. Method step a) is performed in such a way that the electrospinning of the fiber material takes place onto the surface of a carrier element arranged in an electrospinner. The carrier element is an, in particular flat, element, which is detachably arranged on a collector of an electrospinner and which serves the purpose of catching and carrying the spun fibers. The surface of the carrier element is a defined surface, that is, a surface of specified size and geometry. Method step a) is further performed in such a way that a fiber layer with a fiber number (here also referred to as fiber density) of 1000 to 30000 fibers/mm² is produced on the defined surface of the carrier element. Method step a) thus produces the fiber layer and simultaneously applies it to the surface of a carrier element. The invention provides for providing an electrospinning process with a defined material onto a defined surface so that an exactly defined particularly low fiber density is achieved. A very thin fiber layer with low fiber density is thus provided in a particularly advantageous manner, which, in the course of the subsequent method steps, is contacted with basement membrane protein-secreting cells, which deposit the basement membrane proteins in and onto the fiber layer and create a matrix, which embeds the fiber layer and the thickness of which lies in the magnitude of natural basement membranes.

In a method step b), a stabilizing frame is applied to the fiber layer located on the carrier element after application of the fiber layer according to method step a). Method step b) is preferably performed in or by means of a polymer application device, in particular in a polymer application device, which is separate from the electrospinner. In one embodiment, method step b) can also be performed in the electrospinner by means of a polymer application device. The stabilizing frame also serves the purpose of being capable of removing the fiber layer according to the invention from the surface of the carrier element without damage and in a dimensionally stable manner and of thus ensuring a mechanical stabilization, in particular during the removal.

The application of the stabilizing frame onto the fiber layer takes place under conditions, which allow for an associating of the stabilizing frame with the fiber layer. In connection with the present invention, an “associating” is a, preferably reversible, binding of the stabilizing frame to the fiber layer, which takes place in particular by means of adhesion and which is strong enough to provide for a detaching, which is preferably provided in a method step v), of the fiber layer associated on the stabilizing frame from the surface of the carrier element. In one embodiment of the invention, the binding of the stabilizing frame to the fiber layer can also be an irreversible binding.

In a following method step c), the fiber layer is cultivated with cells of at least one cell type, which are introduced into the fiber layer, wherein this cell type is capable of expressing at least one basement membrane protein, in particular of expressing and secreting at least one basement membrane protein into the cell environment. In method step c), the invention thus provides for introducing or applying, respectively, cells of the at least one cell type into and onto the fiber layer, thus for bringing the fiber layer associated with the stabilizing frame into contact with cells of the at least one cell type and for cultivating the cells in the fiber layer. In one embodiment, the construct of fiber layer and stabilizing frame, which is connected to the carrier element, can also be brought into contact with cells of the at least one cell type, the cells can be cultivated in the fiber layer and can be subjected to the further method steps, optionally under detachment of the carrier element, which takes place then.

Method step c) is performed under conditions, which allow for the expression of said at least one basement membrane protein in the cell culture container, in particular which allow for the expression and secreting of the basement membrane protein from the cell into its environment and thus into the fiber layer, so that a fiber layer is obtained in method step c), in the intermediate fiber spaces of which and on the fiber surfaces of which basement membrane proteins secreted from the cells are embedded and supported. These basement membrane proteins thus form an extracellular matrix with fiber layer embedded therein.

According to the invention, it is thus accordingly made possible in an advantageous manner to provide a physiological protein composition and structure, which is as natural as possible and which largely corresponds to the protein composition and structure of natural basement membranes. According to the invention, this protein component of the basement membrane, which is created naturally, is located in particular in the intermediate spaces and on the surfaces of the electrospun material, that is, in and on the fiber layer, and thus forms an integral hybrid structure of synthetically produced fibers and naturally produced basement proteins surrounding said fibers, with this fiber layer. The cells of the at least one cell type are thus particularly preferably cultivated within the open and thin fiber layer structure, which was obtained according to method steps a) and b), and thus also secrete the basement membrane proteins within the fiber layer and not solely on a surface thereof. According to the invention, it thus becomes possible to obtain a basement membrane, the protein components of which do not rest on the surface of a synthetic fiber layer but wherein the fiber layer is integral part of the basement membrane protein structure and the basement membrane proteins are thus also present within the fiber layer and which thus has only a small thickness as a whole.

With regard to their modifications, such as glycosylations, the basement membrane proteins provided according to the invention, just like the proteins occurring in natural basement membranes, can correspond exactly to the naturally occurring basement membrane proteins. According to the invention, in particular preferred cells are used, which produce reticular basement membrane proteins, such as type 4 collagen, laminins and fibronectins. The cultivation of cells of this type on the fiber layers over a time, which is sufficient for expressing and secreting the desired proteins, allows for the synthesizing of proteins, which are of a completely biological and thus natural origin, within the electrospun, in particular thin, fiber layer, and wherein said proteins can be produced without the use of animal materials and media.

The cells of the at least one cell type are removed in a following method step d), so that the biologically produced matrix and the synthetically produced fiber layer embedded in said matrix and thus the basement membrane according to the invention remain and are preserved.

The method according to the invention advantageously provides the basement membrane according to the invention by directly using living cells for introducing naturally produced basement membrane proteins into the, preferably very thin, fiber layer, so that the basement membrane is very similar to a naturally occurring basement membrane and the small thickness thereof, in particular due to the basement membrane proteins, which are produced in vitro.

The method according to the invention advantageously allows producing a particularly thin basement membrane, keeping the amount of artificial, thus synthetic material used for the production as low as possible and obtaining a particularly high percentage of natural basement membrane protein to synthetic material by means of the expression of the natural basement membrane proteins provided in the method.

The fiber material used in method step a) is a material, which can be electrospun, in particular a polymer, in particular a synthetic or a natural polymer. Advantageously and in preferred embodiment, the fiber material is water-insoluble. In particularly preferred embodiment, the fiber material is biocompatible, that is, in particular that it does not negatively impact the growth and the functionality of cells, in particular the ability thereof of expressing proteins and of secreting them.

In a particularly preferred embodiment, the fiber material is not biodegradable.

In a particularly preferred embodiment, the fiber material is biodegradable.

In a particularly preferred embodiment, it is provided that the fiber material used in method step a) is selected from the group consisting of non-degradable polymers, in particular polyether, polyamides, polyacrylonitrile, polystyrene, polyethylene terephthalate, polyoxazoline, chemically functionalized polysaccharides and (block) copolymers thereof, degradable polymers, in particular polyester, poly-α-hydroxy acids, polyglycolic acid, polylactides, polybutiric acids, polyurethanes, polypeptides and (block) copolymers thereof, natural polymers, in particular polysaccharides, plant, animal and human proteins, lecithins and inorganic materials, in particular condensed tetraethyl orthosilicate or condensed metal alcoholates, silica gel, SiO₂, TiO₂, bioglass and carbon. In one embodiment, combinations of the mentioned substances can also be spun.

In a particularly preferred embodiment, it is provided that the fiber material is polyamide, poly-α-hydroxy acid or polyacrylonitrile.

In a particularly preferred embodiment, the fibers can have a diameter of 200 to 1500, in particular 300 to 1000, in particular 300 to 600 nm.

According to the invention, the individual fibers of the fiber layer are freely movable in relation to one another and are not connected to one another in particularly preferred embodiment.

The electrospinning of the at least one fiber material in method step a) can preferably be performed with a voltage of 6 kV to 25 kV, in particular 8 to 20 kV. The relative humidity (at room temperature) during the electrospinning can preferably be 10 to 90%, in particular 20 to 80%, in particular 25 to 50%, can in particular be set in this way.

In preferred embodiment, additional polymers for the electrospinning of the at least one fiber material in method step a) can be electrospun simultaneously and/or alternately via separate nozzles or cannulas with suitable positioning. Additional polymers can furthermore be dissolved directly in the solution of the at least one fiber material in method step a) and can be spun as polymer mixture.

In a particularly preferred embodiment of the invention, it is provided that method step a), that is, the electrospinning, takes place over a timer period of 1 to 30, preferably 2 to 25, in particular 3 to 20 min based on a fiber source.

In a particularly preferred manner, it can also be provided to set the amount of material applied per area of the carrier element in a targeted manner, in particular by variation of the parameter area, spinning time, conveying rate and concentration of the polymer in the spinning solution.

In preferred embodiment, the amount of material of the fibers applied to the surface of the carrier element is 10 to 150 μg/cm², preferably 18 to 70 μg/cm² (in each case based on surface of the carrier element).

In a preferred embodiment, the fiber layer has meshes with an average mesh area of maximally 400 μm², in particular maximally 200 μm², in particular maximally 150 μm².

In further preferred embodiment, the average mesh area of the fiber layer lies in a range of 10 to 400 μm², preferably 10 to 200 μm², preferably 10 to 150 μm², preferably of 25 to 120 μm².

In further preferred embodiment, the average mesh area of the fiber layer lies in a range of 10 to 400 μm², preferably 10 to 200 μm², preferably 10 to 150 μm², preferably of 25 to 120 μm².

In preferred embodiment, the average mesh area is in particular 20 to 200 μm², in particular 50 to 200 μm², in particular 100 to 200 μm², in particular 150 to 200 μm², in particular 10 to 150 μm², in particular 20 to 150 μm², in particular 50 to 150 μm², in particular 100 to 150 μm², in particular 10 to 100 μm², in particular 20 to 100 μm², in particular 50 to 100 μm², in particular 10 to 50 μm², in particular 20 to 50 μm².

In particularly preferred embodiment the invention provides that the fiber number per area is from 1000 to 30000 fibers per mm², in particular 1500 to 25000 fibers per mm², in particular 2000 to 20000 fibers per mm².

In a particularly preferred embodiment, it is provided that the fiber layer has an average mesh area of 10 to 200 μm² or that the amount of material of the fibers applied to the surface of the carrier element is 10 to 150 μg/cm² surface of the carrier element, or both.

In a particularly preferred embodiment, it is provided that the fiber layer has a thickness of 1 to 20 μm, preferably 1.5 to 10 μm, in particular 1 to 5 μm, in particular 1 to 3 μm, in particular 1.5 to 3 μm, in particular up to 1 to 2 μm or 2 to 3 μm. In a particularly preferred embodiment, it is provided that the fiber layer has a thickness of 1 to 3 μm.

In a particularly preferred embodiment, it is provided that without considering optionally available stabilizing fibers, the fiber layer has a thickness of 1 to 20 μm, preferably 1.5 to 10 μm, in particular 1 to 5 μm, in particular 1 to 3 μm, in particular 1.5 to 3 μm, in particular up to 1 to 2 μm or 2 to 3 μm. In a particularly preferred embodiment, it is provided that without considering optionally available stabilizing fibers, the fiber layer has a thickness of 1 to 3 μm.

In a particularly preferred embodiment, it is provided that in consideration of optionally available stabilizing fibers, the fiber layer has a thickness of 1 to 20 μm, preferably 1.5 to 10 μm, in particular 1 to 5 μm, in particular 1 to 3 μm, in particular 1.5 to 3 μm, in particular up to 1 to 2 μm or 2 to 3 μm. In a particularly preferred embodiment, it is provided that in consideration of optionally available stabilizing fibers, the fiber layer has a thickness of 1 to 3 μm.

In a particularly preferred embodiment, the carrier element is a flat metal structure, in particular metal foil, in particular an aluminum foil.

In particularly preferred embodiment, it can be provided that the surface of the carrier element, which is to be webbed, is coated with a modification material, in particular a water-soluble material. This can advantageously have the effect that an electrostatic adhesion of the electrospun fibers to the carrier element is prevent or reduced in this way.

In a particularly preferred embodiment, it is provided that the surface of the carrier element is coated with a water-soluble modification material in a method step x) prior to method step a).

In a particularly preferred embodiment, the water-soluble modification material can be a water-soluble polymer, a water-soluble saccharide or a water-soluble inorganic material.

In particularly preferred embodiment, the water-soluble modification material can be a coating, a film or a particulate coating. The water-soluble modification material can in particular be a water-soluble polymer, in particular polyvinyl alcohol, polyethylene glycol, polyvinyl pyrrolidone, polyacrylamide, polyacrylic acid or a mixture thereof. In particularly preferred embodiment, the water-soluble modification material can also be a water-soluble saccharide, for example oligosaccharide. In further preferred embodiment, the water-soluble modification material can also be an inorganic material, for example a water-soluble salt, in particular sodium chloride or other sodium, potassium and nitrate salts.

In a particularly preferred embodiment, it is provided that following method step a), the carrier element having the fiber layer is removed from the electrospinner in a method step z) and is transferred into a device for performing method step b).

In a particularly preferred embodiment, the stabilizing frame is formed as cell culture container in method step b). In preferred embodiment, a stabilizing frame is thus applied in method step b), which is formed, configured and suitable to allow a cultivation of cells together with the fiber layer. In this embodiment, the stabilizing frame serves the purpose of cultivating the cells of the at least one cell type in the subsequent method c).

In a particularly preferred embodiment, the stabilizing frame is formed as cell culture container in method step b) and the cultivation of the cells provided in method step c) is performed in the stabilizing frame, which is formed as cell culture container.

The device for performing method step b) is preferably a polymer application device, in particular a printer or sprayer.

The stabilizing frame is preferably composed of one or several polymers. In a particularly preferred embodiment, the polymer is a biocompatible polymer, in particular a thermoplastic, in particular polycaprolactone (PCL), polyetheretherketone (PEEK), polylactide (PLA), polyetherimide (PEI), PE (polyethylene), PP (polypropylene), PC (polycarbonate), PS (polystyrene), PET (polyethylene terephthalate), PA (polyamide), POM (polyoxymethylene), PGA (polyglycolic acid), PHB (polyhydroxy butyric acid) or poly(methyl methacrylate) (PMMA) or a combination thereof.

In a particularly preferred embodiment, it is provided that the stabilizing frame is applied to the fiber layer by means of a printing method in method step b), in particular to the top side thereof, thus the side facing away from the surface of the carrier element.

In a particularly preferred embodiment, the printing method can take place using a 3D printer.

In a particularly preferred embodiment, the material of the stabilizing frame can be applied to the fiber layer by means of a fused deposition modeling (FDM), preferably utilizing thermoplastics, in particular biocompatible materials, for the three-dimensional printing.

According to the invention, it is preferred that the stabilizing frame applied in method step b) is individually adapted to the size and shape of the application intended for the produced artificial basement membrane. In preferred embodiment, the stabilizing frame can have the shape and size of a transwell system, of a bioreactor or of an organ/human-on-a-chip system. In preferred embodiment, the stabilizing frame can be ring-shaped, thus for example a stabilizing ring, or angular, in particular rectangular.

In particularly preferred embodiment, the stabilizing frame has a width of 100 to 1000 μm, preferably 200 to 800 μm, and a thickness of 50 to 600 μm, in particular 100 to 500 μm.

In a particularly preferred embodiment, the fiber layer associated with the stabilizing frame is detached from the carrier element in a method step v) at a time after method step b), in particular prior to performing method step c), after performing method step c), prior to performing method step d) or after performing method step d).

The detachment of the stabilizing frame with the adhering fiber layer from the carrier element, which is preferably provided in method step v), can in particular take place by incubation in aqueous solution, in particular water, for example for 0.5 to 5 minutes, in particular 0.5 to 1 min.

In preferred embodiment, it is provided in a method step v) that a detachment of the fiber layer associated with the stabilizing frame from the surface of the carrier element initially takes place prior to the cultivation of the fiber layer associated with the stabilizing frame in method step c).

In a particularly preferred further embodiment, the transfer of the fiber layer associated with the stabilizing frame into a cell culture container takes place in a method step following method step b), here also referred to as method step w). Depending on the device, in which the application of the stabilizing frame has taken place, the transfer takes place either from the electrospinner or from the polymer application device.

In preferred embodiment, it is provided in a method step v) that prior to the transfer of the fiber layer associated with the stabilizing frame into the cell culture container provided in method step w), a detachment of the fiber layer associated with the stabilizing frame from the surface of the carrier element takes place initially, so that only the fiber layer fixed to the stabilizing frame is transferred, without the carrier element.

Following the detachment of the fiber layer connected to the stabilizing frame from the carrier element provided in method step v), this construct is preferably transferred into a cell culture container in a method step w) and is cultivated with cells of at least one cell type, which are introduced into the fiber layer in the following method step c) in the cell culture container, wherein this cell type is capable of the expression of at least one basement membrane protein, in particular of the expression and secretion of at least one basement membrane protein into the cell environment. In one embodiment, the construct of fiber layer and stabilizing frame, which is connected to the carrier element, can also be transferred into the cell culture container and can be subjected to the further method steps, optionally under detachment of the carrier element, which takes place then.

In a particularly preferred embodiment, the fiber layer associated with the stabilizing frame is thus transferred into a separate cell culture container in a method step w) following method step b) and method step c) is performed in the separate cell culture container.

In a further preferred embodiment, it can be provided to stabilize the fiber layer on one of its sides, in particular top or bottom side.

In a particularly preferred embodiment, it is provided that in a method step y) prior to method step c), stabilizing fibers with a fiber diameter of 1 to 500 μm of a stabilizing material are applied to at least one side, in particular the top side or bottom side or both sides of the fiber layer.

In a further preferred embodiment, it can be provided to stabilize the fiber layer on its bottom side by applying stabilizing fibers, that is, on the side, on which the stabilizing frame does not rest.

In a further preferred embodiment, it can be provided to stabilize the fiber layer on its top side by applying stabilizing fibers, that is, on the side, on which the stabilizing frame does rest.

In particularly preferred embodiment, the fiber diameter of the stabilizing fibers can lie in a range of 1 to 500 μm, in particular 5 to 200 μm, in particular 5 to 20 μm.

In particularly preferred embodiment, the stabilizing fibers can consist of polyamide, polycaprolactone or polyester or can comprise them.

In particularly preferred embodiment, commercially available nets, in particular made of polyamide or polyester, can be applied.

In particularly preferred embodiment, the stabilizing fibers can be applied by means of direct application of the fibers by means of microfiber spinning method, for example melt electrowriting, pressure spinning, melt blow spinning or fiber drawing.

In a particularly preferred embodiment, it is provided that the stabilizing fibers are applied to the top side of the fiber layer located on the carrier element in a method step y1) following method step a) and prior to method step b).

In a particularly preferred embodiment, it is provided that the stabilizing fibers are applied to the bottom side of the fiber layer in a method step y2) following the detachment of the fiber layer associated on the stabilizing frame from the carrier element performed in method step v) and prior to the transfer into a cell culture container, which is preferably provided in method step w).

In particularly preferred embodiment, the cells of at least one cell type cultivated in method step c) are cells, which are capable of expressing and secreting proteins of the basement membrane, in particular structural proteins, in particular proteoglycans, in particular type 4 collagen, laminins, fibronectin or a combination thereof.

In particularly preferred embodiment, the cells of at least one cell type are cells, which are capable of expressing and secreting fibrillar structural proteins, such as type 1, 3 or type 7 collagen.

In particularly preferred embodiment, the human cell lines providing fibrillar collagens can be MCF-8, HeLa, HUVEC, HEK203, L929, A549, Caco-2 or MDCK.

In a particularly preferred embodiment, the cells providing fibrillar collagens can be human, stromal cell lines, in particular HFF-1, MRC-5, HS-27A, HS-5 or NGH-3T3.

In a particularly preferred embodiment, the cells providing fibrillar collagens can be induced pluripotent stem cells (iPSC), in particular IMR90-4, UKBi005 or UKKi011.

Primary human cell lines can furthermore be used, which are capable of expressing and secreting Col3 (type 3 collagen). Primary human, stromal cell lines, iPSC-derived stromal cells and animal stromal cells, in particular also cell lines, can furthermore also be used for this purpose.

In particularly preferred embodiment, the cells of at least one cell type are cells, which are capable of expressing and secreting reticular structural proteins, such as type 4 collagen, fibronectins or laminins.

In a particularly preferred embodiment, endothelial cells, epithelial cells or tumor cells can be used.

In a particularly preferred embodiment, the cells can be cells of human cell lines, primary human cells, iPSC-derived cells or animal cells.

In a particularly preferred embodiment, the human cell lines can be cell lines from gigablastomas or cell lines from other carcinomas, in particular from breast, lung, prostate, stomach cancer or invasive or metastasizing melanomas. In particularly preferred embodiment, the human cell line can be from the epithelium. In particularly preferred embodiment, the cell line can be from the endothelium.

In a particularly preferred embodiment, the primary human cells can be tumor cells, epithelial or endothelial cells. In a particularly preferred embodiment, the iPSC-derived cells can be tumor, epithelial and/or endothelial cells. In particularly preferred embodiment, the animal cells can also be animal cell lines, in particular tumor cells, epithelial and endothelial cells.

In a particularly preferred embodiment, it is provided that the cell type is selected from the group consisting of human cell lines, animal cell lines, primary human cells, primary animal cells, induced pluripotent cell lines (IPSC), IPSC-derived cells and stromal cells.

In a particularly preferred embodiment, the cultivation takes place for a time period of 5 to 30, in particular 7 to 21 days.

In a particularly preferred embodiment, it is provided that method step c) is performed at least twice sequentially with cells of different cell types.

In particularly preferred embodiment, a method is provided in which a two-layer basement membrane is produced. In particularly preferred embodiment, it can be provided to cultivate cells of at least a first cell type in a first phase and subsequently cells of at least a second cell type in a second phase in a method step c), so that different basement membrane proteins are deposited in the fiber layer in the two different phases.

In a particularly preferred embodiment, it can be provided in a first phase of method step c) to cultivate cells of a first cell type, which provide for a provision of fibrillary proteins, in particular type 3 collagen or type 1 collagen, and, in a second phase, to cultivate cells of a second cell type, which make it possible to provide reticular proteins, in particular type 4 collagen or laminins. Due to the time sequence, a formation of a reticular protein layer above the fibrillary proteins introduced during the first phase thus takes place in the second phase.

In a particularly preferred embodiment, the detaching of the cells of the at least one cell type provided in method step d) can take place by obtaining the artificial basement membrane by incubation in decellularizing solutions, in particular in solutions with the agents sodium dodecyl sulfate, sodium deoxycholate, Triton X-100, tri-n-butyl phosphate (TnBP), CHAPS (3-[(3-cholamidopropyl)dimethylammonium]-1-propansulfonate) or hypertonic or hypotonic solutions. Decellularizing solutions are preferably used, wherein the decellularizing agents are present in a concentration of 0.1 to 3% by volume.

In a particularly preferred embodiment, the incubation time for the decellularization is from 0.5 to 5 hours (h).

In a particularly preferred embodiment, it can be provided to wash out the cell fragments, which may be present in the product produced according to the invention after the decellularization, by rinsing one or several times, in particular with physiological saline solution, and to degrade optionally remaining DNA by means of in particular 0.5- to 2-hour, in particular one-hour, incubation in DNase solution.

In one embodiment of the invention, the stabilization frame can be removed following method step c), in particular following method step d).

In a particularly preferred embodiment, it is provided that the artificial basement membrane has a thickness of 1 to 20 μm, preferably 1.5 to 10 μm, in particular 1 to 5 μm, in particular 1 to 3 μm, in particular 1.5 to 3 μm, in particular 1 to 2 μm or 2 to 3 μm.

In a particularly preferred embodiment, it is provided that the artificial basement membrane has a thickness of 1 to 3 μm.

In a particularly preferred embodiment, it is provided that, without considering optionally available stabilizing fibers, the artificial basement membrane has a thickness of 1 to 20 μm, preferably 1.5 to 10 μm, in particular 1 to 5 μm, in particular 1 to 3 μm, in particular 1.5 to 3 μm, in particular 1 to 2 μm or 2 to 3 μm.

In a particularly preferred embodiment, it is provided that, without considering optionally available stabilizing fibers, the artificial basement membrane has a thickness of 1 to 3 μm.

In a particularly preferred embodiment, it is provided that, in consideration of optionally available stabilizing fibers, the artificial basement membrane has a thickness of 1 to 20 μm, preferably 1.5 to 10 μm, in particular 1 to 5 μm, in particular 1 to 3 μm, in particular 1.5 to 3 μm, in particular 1 to 2 μm or 2 to 3 μm.

In a particularly preferred embodiment, it is provided that, in consideration of optionally available stabilizing fibers, the artificial basement membrane has a thickness of 1 to 3 μm.

The present invention also provides an artificial basement membrane, comprising a fiber layer of electrospun fibers with a fiber diameter of 100 to 2000 nm, wherein the fiber layer is embedded in a matrix created by cells of at least one cell type, which are capable of expressing at least one basement membrane protein, in particular produced by means of a method according to the invention.

The present invention also provides an in vitro test system, cell culture system, artificial tissue, implant, insert system, filtration system and human-on-a-chip/organ system, comprising at least one basement membrane according to the invention.

The present invention in particular provides an in vitro test system, in particular barrier model, comprising at least one basement membrane according to the invention.

The basement membrane according to the invention can be used in a cell-free or cell-containing manner.

According to the invention, the basement membrane can preferably be utilized in a cell-free or cell-containing embodiment as insert system or as organ/or human-on-a-chip system, respectively, in particular applied in the field of the:
blood-brain barrier/neurovascular unit, intestinal/gastrointestinal tract, endothelium, lung, airway, skin, oral mucosa, cornea, retinal pigmented epithelium, pathological variants of the mentioned models, liver, kidney, all further internal organs.

The basement membrane according to the invention can be used in vitro or in vivo, for example as cell-free or cell-containing implant.

The basement membrane according to the invention can also be used as filtration system, in particular filter or filter material, in particular for sterile filtrations.

The basement membrane according to the invention can also be used for the stem cell culture or the culture and application of spheroids, organoids and assembloids.

In a particularly preferred embodiment, the artificial basement membrane comprises cells of at least one target cell type, which are applied in or to the artificial basement membrane.

Cells of a target cell type are cells, which are introduced onto or into a basement membrane according to the invention and which are cultivated in order to thus provide a desired cell-based applicability using cultivated cells, for example as test system, in particular in vitro test system, in particular barrier model or as artificial tissue or organ system.

In preferred embodiment, cells of at least one target cell type can be small intestinal cells, epithelial cells, endothelial cells or tumor cells.

In a preferred embodiment of the invention, the at least one target cell type is selected from the group consisting of fibroblasts, in particular cancer associated fibroblasts, keratinocytes, mesenchymal stem cells, iPSC cells (induced pluripotent stem cells), tumor cells, endothelial cells and combinations thereof.

The invention also relates to the use of a basement membrane according to the invention for the cultivation or differentiation of cells.

The invention also relates to an artificial tissue comprising at least one basement membrane and at least one cell of at least one target cell type.

In a preferred embodiment, the artificial tissue has several, in particular at least two, in particular at least three, basement membranes according to the invention.

The invention also relates to an artificial tissue, comprising a basement membrane according to the invention and cells of at least one target cell type, wherein the artificial tissue comprises 1 to 50% by weight of basement membrane and 50 to 99% by weight of cells, in particular 80 to 99% by weight of cells (in each case based on the total weight of the artificial tissue).

In preferred embodiment, the artificial tissue is a small intestine model.

In preferred embodiment, the artificial tissue is a model of a blood-brain barrier/neurovascular unit.

In preferred embodiment, the artificial tissue is a skin model, in particular comprising a dermis, comprising at least one basement membrane according to the invention. In preferred embodiment, the artificial tissue is a skin model, preferably comprising a subcutis comprising at least one basement membrane according to the invention.

In preferred embodiment, the artificial tissue is a skin model, in particular comprising a dermis, comprising at least one basement membrane according to the invention and a subcutis comprising at least one basement membrane according to the invention, preferably a different basement membrane.

The at least two basement membranes, which are used in the skin model according to the invention, are preferably formed specifically, they each in particular comprise at least one cell of a specific cell type, in particular of a different cell type.

In preferred embodiment, the cells are present so as to be distributed homogenously within the basement membrane according to the invention.

The present invention also relates to therapeutic applications of the basement membranes according to the invention and artificial tissues having said basement membranes.

The present invention also relates to a method for the treatment of, in particular sick humans or animals and products for the use in such a methods.

The basement membrane according to the invention can in particular be used as wound packing, in particular for the treatment of preferably chronic wounds or burns, in particular by introducing, in particular inserting, into wounds, colonization with tissue cells from the wound edges, closing the wound above the basement membrane according to the invention by means of cells, in particular keratinocytes.

The basement membrane according to the invention can also be used as porous tissue, which in vivo provides for the ingrowth of cells during the regeneration after, for example, surgical procedures or resections in the (cancer) treatment.

The present invention in particular also relates to methods for treating, in particular sick, humans or animals using the artificial tissue according to the invention or the basement membrane and the basement membrane or the artificial tissue according to the invention for use in such methods. The artificial tissue according to the invention can in particular be used as ATMP (Advanced Therapy Medicinal Product), in preferred embodiment colonized with tissue-specific cells of a target cell type, wherein the use in or as stromal tissue/organ(s) is preferred, in particular in or as skin, cartilage, arteries and/or veins, heart, kidney, liver, urogenital tract, airways, bones, intestine or cornea.

The present invention also relates to non-therapeutic applications of the basement membrane according to the invention and artificial tissues having said basement membrane, for example cosmetic applications.

In connection with the present invention, the term “fiber number/mm²” is understood to be the number of fibers per mm² of fiber layer, here also referred to as fiber density, wherein the area refers to the entire area of the top or bottom side of the fiber layer in its flat expansion directions, thus length and width. Only the top side or the bottom side is used for the area calculation, thus not both and also not the edge areas. The fiber density is preferably determined by means of light or scanning electron microscopy.

Expansion directions of spatial structures, such as fiber layer, carrier element or basement membrane of the present invention are referred to as length, width and thickness with reference to a Cartesian three-dimensional coordinate system, wherein the length of the x axis, the width of the y axis lying in the same plane and the thickness corresponds to the z axis standing perpendicularly, in particular vertically, on this plane spanned from x and y axis. Flat expansion directions of the mentioned spatial structures, in particular length and width, are directions, in which the expansion is significantly larger, in particular multiple times larger, in particular at least 5-times larger than in expansion directions, which are not flat, in particular thickness.

In connection with the present invention, the “amount of material applied per area of the carrier element” is calculated according to Example 1.

In connection with the present invention, the “mesh area” of the fiber layer is determined by means of microscopy methods, in particular without medium or in water, in particular without mechanical stress. The application of transmitted light microscopy or of 3D methods, such as laser scanning microscopy (LSM) or the similar method of confocal reflection microscopy, is most suitable thereby. From the created 3D image, the individual images of a nanofiber layer were subsequently placed one on top of the other to form a 2D image. The area of the intermediate spaces between the nanofibers is determined subsequently. In particular, the mesh width is determined according to the procedural instructions from Example 4.

In connection with the present invention, the term “fiber layer”, also referred to as web here, is understood to be a sheet material, which consists of disordered fibers or of fibers aligned in a targeted manner, wherein in preferred embodiment, the fibers are not connected to one another, interwoven or intertwined.

In connection with the present invention, the term “side” is understood to be the side of a fiber layer or of a basement membrane, which is formed in a flat manner, in particular the top or bottom side, wherein the edge areas or front areas of the membrane or of the fiber layer are not captured with the term “side” due to their small thickness expansion.

In connection with the present invention, the term “electrospinner” is understood to be a device, which is used for the electrospinning technique in order to produce fibers of a very small diameter from a polymer solution or melt, in particular in that a high voltage is applied in order to pull a polymer liquid drop into a thin stream, which is then converted to a nanofibrous structure by means of evaporation or solidification. An electrospinner comprises in particular a high-voltage source, a syringe pump for conveying the polymer solution with controlled speed, a nozzle, for example needle or capillary, for the exit of the polymer solution and a collector, for example a metal plate or drum for catching and demolding the fibers.

In connection with the present invention, the term “biocompatible” is understood such that a material or a substance does not negatively impact the growth and the functionality of cells, in particular the ability thereof to express proteins and to secrete them, in particular that the material or the substance is accepted by the human or animal body, in particular without a harmful immune response or toxic effect being triggered during contact. Biocompatible materials are preferably not toxic, not immunogenic, not carcinogenic and/or stable or degradable, depending on the application.

In connection with the present invention, the term “biodegradable” is understood such that the respective element, in particular substance, in particular polymer, can be degraded by means of biological processes, in particular naturally occurring biological processes, in particular biological processing naturally occurring in a human or animal body, and thus loses its structural and/or material integrity.

In connection with the present invention, the term “cell culture container” is preferably understood to be a cell culture plate, microplate, petri dish, bioreactor, microfluidic chip, multi-chamber system, roller bottle or cell culture bottle.

The methods according to the invention are characterized by a preferred sequence of method steps x) (optional coating with modification material), a), y1) (optional application of stabilizing fibers to top side of the fiber layer), z) (optional removal from electrospinner), b), v) (optional detachment of the carrier element), y2) (optional application of stabilizing fibers to bottom side of the fiber layer after performing method step v)), c) and d).

The methods according to the invention are characterized by a preferred sequence of method steps x) (optional coating with modification material), a), y1) (optional application of stabilizing fibers to top side of the fiber layer), z) (optional removal from electrospinner), b), v) (optional detachment of the carrier element), y2) (optional application of stabilizing fibers to bottom side of the fiber layer after performing method step v)), w) (transferring into cell culture container), c) and d).

In particularly preferred embodiment, the method steps take place in the order specified in the present disclosure, unless otherwise specified or apparent to a person of skill in the art.

In a particularly preferred embodiment of the present invention, it is provided that no further method steps are performed between the individual explicitly specified method steps.

In connection with the present invention, the term “and/or” is understood such that all members of a group, which are connected by the term “and/or” are described cumulative with one another in any combination as well as alternative to one another. In an exemplary manner, the expression “A, B and/or C” is to be understood to be the following disclosure content: i) (A or B or C), or ii) (A and B), or iii) (A and C), or iv) (B and C), or v) (A and B and C), or vi) (A and B or C), or vii) (A or B and C), or viii) (A and C or B).

In connection with the present invention, individual components or parts of a totality, in particular of the basement membrane according to the invention or of one of its components, which are determined quantitatively in relative form, in particular in percentages, preferably add up to 100% by weight of the respective referenced totality or of the basement membrane, respectively unless otherwise specified or, if referenced, a component thereof.

In connection with the present invention, non-specified decimal places of a decimal number are understood as 0, unless otherwise specified.

Further advantageous designs of the invention follow from the subclaims.

The following examples and the corresponding figures explain the present invention, but without limiting it.

The figures show:

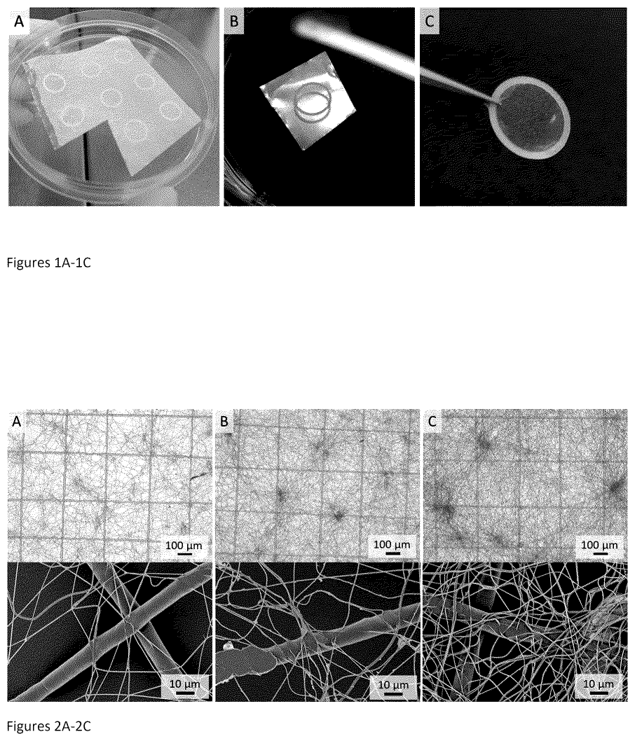

FIGS. 1A-1C handling of the thin fiber layer. FIG. 1A The printing of PCL rings directly onto the fibers took place after production of the thin fiber layer. FIG. 1B The fiber layer with the stabilizing frame can be detached after short incubation in water through the water-soluble intermediate layer of modification material and FIG. 1C can be removed.

FIGS. 2A-2C expansion of the thin fiber layer (polyamide 6) with MEW reinforcement fibers (PCL, polycaprolactone).

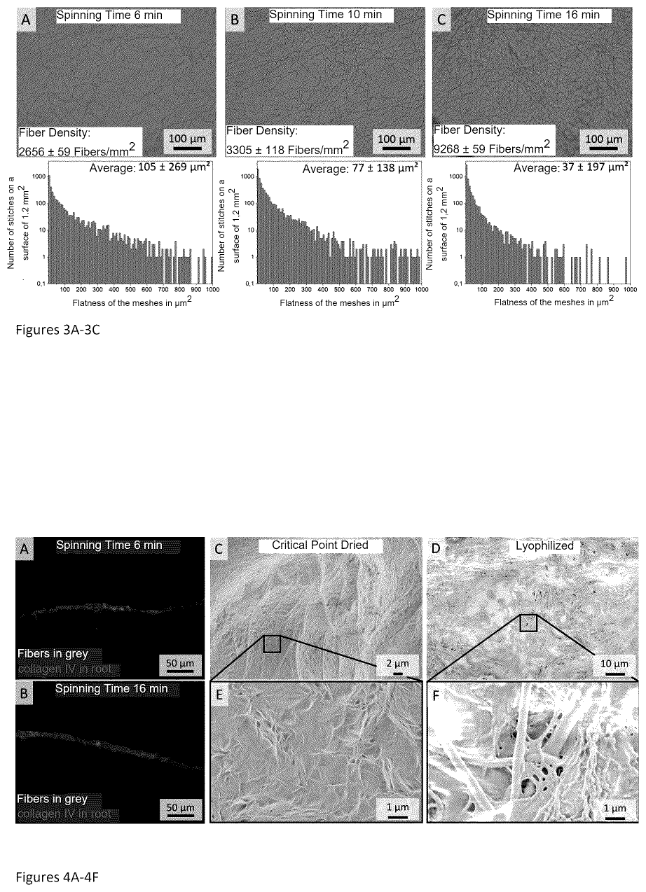

FIGS. 3A-3C characterization of the thin fiber layers by measuring the mesh areas.

FIGS. 4A-4F characterization of the cellularly modified and decellularized fiber layer. In the cross section, the fibers can be recognized by their characteristic shape. The immuno-histologically stained collagen IV can be clearly recognized around and between the fibers.

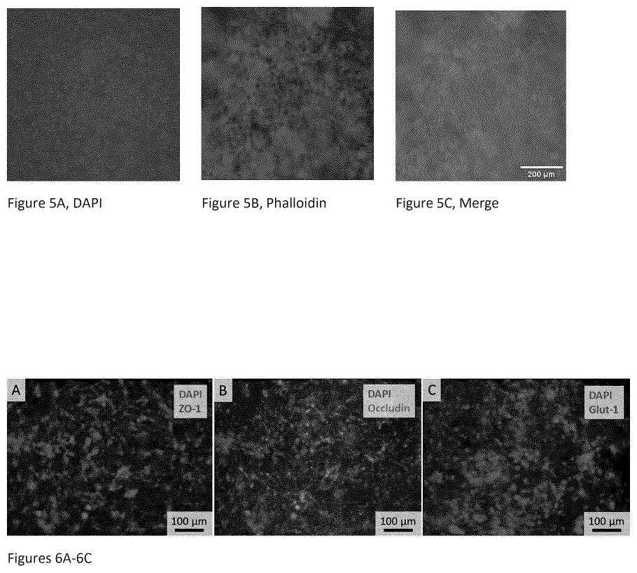

FIGS. 5A-5C small intestinal epithelial cells form monolayers on synthetic basement membrane according to the invention. The DAPI staining FIG. 5A shows the characteristic round cell cores with a diameter of approximately 10 μm. The phalloidin staining FIG. 5B describes the cell body via the cytoskeleton and stains the remainder of the cell except for the cell core. FIG. 5C describes an overlap of both stainings.

FIGS. 6A-6C hiPSC cell-derived endothelial cells of the blood-brain barrier form monolayers on the biologized synthetic basement membrane according to the invention. In all images, the DAPI staining shows the characteristic round cell cores with a diameter of approximately 10 μm. The cytoplasmatic tight junction protein ZO-1 FIG. 6A is present around each cell body in varying presence. A further tight junction protein occludin FIG. 6B describes the connection of the cells and can thus be seen in a line-shaped manner around the individual cells. The membrane transporter protein Glut-1 FIG. 6C is located on the surface of the cells and can thus be recognized on all cells in varying presence.

### EXAMPLES

### Example 1—Basic Structure of Electrospun Fibers and Stabilization Frame

The production of the fiber layers took place in an electrospinner with a rotating roller of the diameter of 10.5 cm. The surface was wrapped with an aluminum foil as carrier element and a defined surface of 396 cm² was set by means of adhesive tape. The coating of the surface with the modification material NaCl powder took place subsequently in order to later simplify the removal of the fibers (method step x)). A polyamide 6-solution with a concentration of 12% m/V in 1,1,1,3,3,3 hexa-fluoro-2-propanol was provided for the spinning process (method step a)) and was placed in a syringe with the distance cannula tip-surface roller of 15 cm. A conveying speed of 0.6 mL/h, a potential difference of 11 kV, a rotational speed of 100 rpm as well as a parallel movement of the cannula to the roller of 10 mm/s was set at the pump during the spinning process. The spinning duration was varied for 6 min, 10 min und 16 min in this example.

The theoretical fiber material content per area is determined from these parameters as follows:

$$conveyed mass / surface = ( 0.12 * 0.6 mL / h * 1.14 g / {cm}^{3} * spinning time ) / 396 {cm}^{2} .$$

The fiber material content per area was: 20.7 μg/cm² (6 min), 34.5 μg/cm² (10 min), 55.3 μg/cm² (16 min).

In a method step z), the fiber layer associated with the stabilizing frame on the carrier element was subsequently removed from the electrospinner and was placed into a FDM 3D printer (Fused Deposition Modeling (FDM)). The printing (method step b)) of the stabilizing rings, thus of the stabilizing frame, took place with polycaprolactone with an inner diameter of 9 mm and outer diameter of 10 mm directly onto the fibers (FIG. 1A). The construct, thus the fiber layer associated with the stabilizing frame, can subsequently be detached from the carrier element by means of short incubation in water and can be removed (method step v)) (Figures. 1B, C).

### Example 2—Additional Stabilization of the Fiber Layer Via μm Fibers

For an additional stabilization, additional fibers can either be applied directly to the electrospun fibers (method step y1)) with following printing of the stabilizing frame or, as described below, after the printing of the stabilizing frame to the bottom side of the construct (method step y2)). For this purpose, the construct of electrospun fibers and frame was released by means of water from the aluminum surface (method step v)), was dried and was placed upside down into an MEW printer (Melt Electrowriting-(MEW)). This was followed by the printing of the electrospun fibers via MEW of polycaprolactone in the form of a checkerboard pattern with fiber-to-fiber distances of 300 μm and a fiber thickness of approx. 10 μm (method step y2)). FIG. 2 shows the resulting structure via different microscopy methods and enlargements. FIG. 2 shows light-microscopic images (top) and REM (scanning electron microscopic) images (bottom) with different electrospun fiber densities, produced with (A) 6 min, (B) 10 min and (C) 16 min spinning time. The reinforcement fibers, which have a significantly larger diameter compared to the fibers of the fiber layer, which are also illustrated, were applied to the rear side of the fiber layer via melt electrowriting.

This approach allows large freedoms in the structure and arrangement of these MEW fibers, in particular with regard to fiber thicknesses, fiber distances, angular orientation of the fibers as well as fibers, which are not placed linearly.

### Example 3—Biologization Process of the Synthetic Basic Structure

The synthetic basic structure, thus the fiber layer, was initially placed into a transwell device (clamped into a cell crown, two plastic cylinders with different diameter, which are placed one inside the other), placed into a 24-well plate (method step w), transfer into cell culture container) and were covered on the outside and on the inside with a total of 1.5 ml of culture medium. This was followed by the cultivation (method step c)) of 100,000 cells per sample for a duration of 14 days (media change 3-times per week). To remove the cells, the medium was initially removed, the sample was covered with a solution of sodium deoxycholate (0.33 g/10 ml VE water; VE: fully desalinated) and was incubated for 30 min at RT on a shaker (method step d)). To remove the cell residues, 4 washing steps took place with PBS (phosphate-buffered saline) for 20 min at RT on a shaker and then the incubation overnight in fresh PBS—at 4° C. The removal of the DNA by means of incubation in a DNAse solution took place in the last step at 37° C. for 1 hour. The synthetic biologized basic structure was stored at 4° C. after an additional 3 washing steps in PBS until the further application. All steps were performed under sterile conditions in order to avoid final tissue-damaging sterilization methods.

### Example 4—Meshes

In order to characterize the fiber densities, the number of fibers/area and the mesh areas were determined by means of scanning electron microscopic or light microscopic images of the fiber layers from Example 1. FIGS. 3A-C (top) show the fiber densities of the three different spinning times in an exemplary manner. FIG. 3A-C, bottom, describes the illustration of the mesh areas. FIG. 3 shows light microscopic images of the fiber layers (top) with corresponding histogram of the mesh areas and number (bottom). The densities of the fiber layers were set via the spinning time of (A) 6 min, (B) 10 min and (C) 16 min. The determination of the fiber density took place manually or the mesh area via ImageJ, respectively, and is illustrated in the size range between 1 μm² and 1000 μm².

### Example 5—Biologized Basement Membrane

In order to analyze the structure and composition of the finished product of the synthetic-based and biologized basement membrane, an immunofluorescence staining against type 4 collagen of paraffin sections took place on the one hand. FIG. 4A shows the fiber layer with a 6 min spinning time and FIG. 4B with a 16 min spinning time. On the other hand, the samples according to FIG. 4B (16 min spinning time) were dried via critical point drying (FIG. 4C, E: for critical point drying with 2 and 1 μm scale) or lyophilization (FIG. 4D, F: for lyophilization with 10 and 1 μm scale) and the tight surface thereof was checked via scanning electron microscopy. The presence of collagen IV (red staining) can be recognized over the complete area and thickness of the fiber layer, by means of immunofluorescence of paraffin sections; in an exemplary manner in the sample with (A) 6 min spinning time and (B) 16 min spinning time. A complete filling of the intermediate fiber spaces with ECM matrix can be confirmed after (C) critical point drying as well as (D) lyophilization (for 16 min spinning time: FIGS. 4C, D, E and F).

### Example 6—Small Intestine

In order to be able to confirm the applicability of the biologized basement membrane according to the invention, human primary small intestinal organoids, cultivated in Matrigel® (3D environment), were disassociated into individual cells with TrypLE Express and 400,000 individual cells were apically applied to the biologized basement membrane. A medium cocktail consisting of a plurality of growth factors, which support the proliferation, suppress the apoptosis and prevent the differentiation is used for a time period of 5 days for the cultivation of the primary organoids, until a closed epithelial cell layer is created. The application of a further medium cocktail (daily media change), which induces a differentiation of the intestinal epithelium in a targeted manner (Schweinlin et al., 2016, DOI: 10.1089/ten. TEC.2016.0101, Däullary et al., 2023, https://doi.org/10.1080/19490976.2023.2186109) takes place for an additional 5 days. The determination of the formation of a closed and single-layer cell layer has been shown using immunofluorescence staining. The cell cores were stained by means of DAPI (blue) and the cell membrane was stained by means of phalloidin (red) (see FIG. 5). FIG. 5 shows representative images of an immunofluorescence staining. The markers (A) DAPI (blue, cell cores) and (B) phalloidin (red, cell membrane) show formation of a single-layer, closed epithelial cell layer on the decellularized basement membrane. (C) shows a merge of the two stained cell structures.

### Example 7—Blood-Brain Barrier

The setup of in vitro models/test systems of the human blood-brain barrier/neurovascular unit represents a further application example of the biologized basement membrane according to the invention. For this purpose, endothelial cells of the blood-brain barrier obtained and differentiated from hiPSC cells (human iPSC) were applied on day 8 of the differentiation A DAPI B phalloidin C merge (Appelt-Menzel et al., 2017, DOI: 10.1016/j.stemcr.2017.02.021) as individual cells in a defined sowing density of 1×10⁶ cells/cm² cultivation area to the biologized basement membrane. The cells were treated for 2 days with cell-specific medium in order to ensure the formation of a tight, barrier-forming monolayer. In order to characterize the differentiation success of the hiPSC cell-derived endothelial cells of the blood-brain barrier, immunofluorescence stainings took place on tissue-specific tight junction proteins and transporters (FIGS. 6A-C). FIG. 6 shows representative images of an immunofluorescence staining for the markers (A) ZO-1 (Zona occludens), (B) occludin and (C) glut-1 (glucose transporter), which show the formation of a monolayer on the biologized basement membrane and the expression of characteristic blood-brain barrier markers.

1. A method for the production of an artificial basement membrane comprising a fiber layer, which is embedded in a matrix composed of at least one basement membrane protein, comprising the following method steps:
a) producing and applying a fiber layer, comprising fibers with a fiber diameter of 100 to 2000 nm, by means of electrospinning a fiber material onto the surface of a carrier element arranged in an electrospinner, wherein the fiber layer has a fiber number of 1000 to 30000 fibers/mm²,
b) applying a stabilizing frame of at least one polymer onto the fiber layer arranged on the carrier element under conditions, which allow for an association of the stabilizing frame with the fiber layer,
c) cultivating the fiber layer associated on the stabilizing frame with cells of at least one cell type, which are introduced into the fiber layer and which are capable of expressing at least one basement membrane protein, under conditions, which allow for the expression of the at least one basement membrane protein and by obtaining a matrix, which is composed of the at least one basement membrane protein and in which the fiber layer is embedded and
d) removing the cells of the at least one cell type by obtaining the artificial basement membrane.

2. The method according to claim 1, wherein the stabilizing frame is formed as cell culture container in method step b) and the cultivation of the cells provided in method step c) is performed in the stabilizing frame, which is formed as cell culture container.

3. The method according to claim 1, wherein the fiber layer associated with the stabilizing frame is transferred into a separate cell culture container in a method step w) following method step b) and method step c) is performed in the separate cell culture container.

4. The method according to claim 1, wherein the fiber layer associated with the stabilizing frame is detached from the carrier element in a method step v) at a time after method step b).

5. The method according to claim 1, wherein the fiber layer has an average mesh area of 10 to 200 μm² or the amount of material of the fibers applied to the surface of the carrier element is 10 to 150 μg/cm² surface of the carrier element, or both.

6. The method according to claim 1, wherein the surface of the carrier element is coated with a water-soluble modification material in a method step x) prior to method step a).

7. The method according to claim 1, wherein the stabilizing frame is applied to the fiber layer by means of a printing method in method step b).

8. The method according to claim 1, wherein following method step a), the carrier element having the fiber layer is removed from the electrospinner in a method step z) and is transferred into a device for performing method step b).

9. The method according to claim 1, wherein in a method step y) prior to method step c), stabilizing fibers with a fiber diameter of 1 to 500 μm of a stabilizing material are applied to at least one side of the fiber layer.

10. The method according to claim 9, wherein the stabilizing fibers are applied to the top side of the fiber layer located on the carrier element in a method step y1) following method step a) and prior to method step b).

11. The method according to claim 9, wherein the stabilizing fibers are applied to the bottom side of the fiber layer in a method step y2) following the detachment of the fiber layer associated on the stabilizing frame from the carrier element provided in method step v) and prior to the cultivation provided in method step c).

12. The method according to claim 1, wherein method step c) is performed at least twice sequentially with cells of different cell types.

13. The method according to claim 1, wherein the fiber material is selected from the group consisting of non-degradable polymers, in particular polyether, polyamides, polyacrylonitrile, polystyrene, polyethylene terephthalate, polyoxazoline, chemically functionalized polysaccharides and (block) copolymers thereof, degradable polymers, in particular polyester, poly-α-hydroxy acids, polyglycolic acid, polylactides, polybutiric acids, polyurethanes, polypeptides and (block) copolymers thereof, natural polymers, in particular polysaccharides, plant, animal and human proteins, lecithins and inorganic materials, in particular condensed tetraethyl orthosilicate or condensed metal alcoholates, silica gel, SiO₂, TiO₂, bioglass and carbon.

14. The method according to claim 1, wherein the cell type is selected from the group consisting of human cell lines, animal cell lines, primary human cells, primary animal cells, induced pluripotent cell lines (IPSC), IPSC-derived cells and stromal cells.

15. The method according to claim 1, wherein the artificial basement membrane has a thickness of 1 to 20 μm.

16. An artificial basement membrane, comprising a fiber layer of electrospun fibers with a fiber diameter of 100 to 2000 nm, wherein the fiber layer is embedded in a matrix created by cells of at least one cell type, which are capable of expressing at least one basement membrane protein, in particular produced by means of a method according to claim 1.

17. The artificial basement membrane according to claim 16, comprising cells of at least one cell target type applied in or to the artificial basement membrane according to claim 16.

18. An in vitro test system, cell culture system, artificial tissue, implant, insert system, filtration system and human-on-a-chip/organ system, comprising at least one basement membrane according to claim 16.
